(19) United States
(12) Patent Application Publication
(10) Pub. No.: US 2021/0322697 A1
(43) Pub. Date: Oct. 21, 2021

(54) MEASURING DEVICE AND METHOD FOR DETERMINING AT LEAST ONE RESPIRATORY PARAMETER

(71) Applicant: FRAUNHOFER-GESELLSCHAFT ZUR FÖRDERUNG DER ANGEWANDTEN FORSCHUNG E. V., München (DE)

(72) Inventors: Jan RINGKAMP, Stuttgart (DE); Jens LANGEJÜRGEN, Stuttgart (DE); Philipp LEBHARDT, Stuttgart (DE); Philipp RADLER, Stuttgart (DE)

(73) Assignee: FRAUNHOFER-GESELLSCHAFT ZUR FÖRDERUNG DER ANGEWANDTEN FORSCHUNG E. V., München (DE)

(21) Appl. No.: 16/973,736

(22) PCT Filed: Jun. 19, 2019
(86) PCT No.: PCT/EP2019/066257

§ 371 Date: Dec. 9, 2020

(30) Foreign Application Priority Data
Jun. 20, 2018 (DE) 10 2018 210 051.9

(51) Int. Cl. A61M 16/00; A61B 5/08; A61B 5/05

(52) U.S. Cl. CPC A61M 16/024; A61B 5/0809; A61B 5/05; A61M 16/0003; A61M 5/007; A61M 2230/42; A61M 2230/46; A61M 2016/0018; A61M 2205/3317

(57) ABSTRACT

The invention relates to a measuring device and to a method for determining at least one respiratory parameter, wherein an electromagnetic field is irradiated into a body and is received on an opposite side of the body, then a phase of the received alternating field is compared in a time-dependent manner to a phase of the irradiated alternating field, and the at least one respiratory parameter is determined from a result of the comparison.

The invention relates to a measuring device and to a method for determining at least one respiratory parameter, wherein an electromagnetic field is irradiated into a body and received on an opposite side of the body, then a phase of the received alternating field is compared to a phase of the irradiated alternating field as a function of time, and the at least one respiratory parameter is determined from a result of the comparison.

Monitoring respiration has a multitude of practical applications. For example, the measurement of respiratory activity, for example in special situations or when exercising, plays a central role in a number of scientific examinations. Such examinations provide a better understanding of the respiratory behavior as a function of outside influences.

Monitoring respiration can also be important for the mechanical ventilation of patients. Here, it should be avoided, for example, to carry out artificial respiration in place of spontaneous breathing.

A majority of the established methods combine an electrocardiogram (ECG) with impedance cardiography (ICG) and estimate the cardiac component and subtract it from the impedance signal to obtain the respiratory component from the resulting signal. Since the detection of the respiratory parameters is indirectly derived through the estimation of the cardiac component, it is necessarily dependent on the quality of the cardiac model. Moreover, primarily the respiratory frequency is determined in the process. It is not possible to provide direct information about the tidal volume and spontaneous breathing.

Another method is the measurement of the expansion of the chest and of the abdomen using respiratory inductive plethysmography (RIP). Here, two elastic bands are placed around the patient's thorax and abdomen. Coils are incorporated into the bands, which change the inductance thereof with an expansion of the bands. This method may seem unpleasant and constraining to the patient, due to the elastic bands. A comparable method is also conceivable by way of strain gauges, resulting in no further advantages of the method.

It is the object of the present invention to provide a measuring device and a method for determining at least one respiratory parameter, which allow the corresponding parameter to be determined, while nonetheless being comfortable to wear and not restricting the mobility of the person wearing it.

This object is achieved by the measuring device for determining at least one respiratory parameter according to claim 1, by the method for measuring at least one respiratory parameter according to claims 10 and 12, and by the respiratory apparatus according to claim 24, the device for injecting a contrast agent according to claim 25, and by the device for imaging according to claim 26. The respective dependent claims provide advantageous embodiments of the measuring device according to the invention and of the method according to the invention.

According to the invention, a measuring device for determining at least one respiratory parameter is provided. The measuring device comprises at least one transmitting structure and at least one receiving structure. Advantageously, the at least one transmitting structure can be an antenna or an electrode. The at least one receiving structure can preferably be an antenna or an electrode.

The at least one transmitting structure and/or the at least one receiving structure are preferably designed so as to be attachable to the outside of a body of a person, of an animal or of a phantom. The transmitting structure and/or the receiving structure can, for example, be designed to be adhesive. An electrically conductive contact between the transmitting structure and/or receiving structure and the body can be advantageous, but is not necessary.

The measuring device according to the invention comprises at least one signal generator, which is electrically coupled to the at least one transmitting structure. An AC voltage, which can be applied to the transmitting structure, can be generated by the signal generator. In this way, an alternating electromagnetic field can be generated by the transmitting structure.

In an advantageous embodiment of the invention, the antennas of the receiving and transmitting structures are arranged so as to be alternately located in the near field. The antenna of the transmitting structure is then coupled to the corresponding antenna of the receiving structure within the meaning that a coupled impedance forms between the antennas. This impedance Z₁₂ can be described as follows for the example of two dipoles serving as the transmitting and receiving antennas:

$${Z}_{12} = \frac{{l}^{2}}{4 π {r}^{2}} [ \frac{1}{\underbrace{j ω \cdot ɛ \cdot r}_{C}} + {R}_{0} + \underbrace{j ω \cdot µ \cdot r}_{L} ] {e}^{- j 2 π r / λ}$$

In the process the permittivity term prevails, the closer in relation to the wavelength the antennas are (H. Wheeler, “The Radiansphere around a Small Antenna,” Proc. IRE, vol. 47, no. 8, pp. 1325-1331, 1959). If the measurement is based on a detection of the change in permittivity, for example, it is consequently advantageous to have the largest possible wavelength λ, so as to keep the ratio of r (distance between the antennas) to λ (wavelength) as small as possible. Advantageously, an optimal wavelength (or frequency) is selected, which in relation to the body is small enough to be able to see a clear shift of the phase during inspiration and expiration, yet large enough to still obtain significant coupling within the aforementioned meaning. The invention, however, can also be carried out when the antennas are not located in the near field, that is, are not coupled within the aforementioned meaning.

If the antennas are to be arranged in the near field with respect to one another, it is advantageous when the distance between the antennas is smaller than or equal to 4 times the wavelength λ, advantageously smaller than or equal to 3 times the wavelength λ, and advantageously smaller than or equal to twice the wavelength λ. Optionally, the definition of the near field of the IEEE may serve as a basis. Here, the outer boundary is defined as the distance λ/(2π) from the antenna surface, where λ is the wavelength in the free space (see IEEE Standard for Definitions of Terms for Antennas, IEEE Std 145-2013). Consequently, the distance between the antennas of the antenna pair is advantageously smaller than or equal to λ/(2π).

The coupled system of the transmitting and receiving structures is advantageously operated in the resonant frequency of the transmitting antenna and/or the resonant frequency of the resonant frequency created in the transmitting antenna by the receiving antenna.

It is advantageous when the transmitting and receiving antennas each form a shared resonance due to the coupling within the aforementioned meaning. The quality of the resonance can particularly advantageously be adapted or varied so as to be adapted to the specific application. In the process, a high quality results in high sensitivity with a smaller dynamic range. Correspondingly, a low quality results in low sensitivity with a larger dynamic range. Here, dynamics is defined as the measurement range that can be unambiguously assigned to a phase offset [0 . . . 2Pi]. If the phase offset extends across more than 2*Pi, the phase offsets no longer unambiguously correspond to a distance between the antennas. This is not necessarily problematic since it is also possible to identify the distance between the antennas based on the progression of the phase offset; however, it is advantageous to select the wavelength in such a way that the range 0 to 2Pi is not exceeded when the respiration runs through the full amplitude.

For example, a high quality may be suitable for use on newborn or premature babies since only a small volume range has to the measured, except at a high resolution. A low quality may be suitable for use on adults since a large volume range has to be measured, but a lower resolution is sufficient.

In an advantageous embodiment of the invention, the transmitting and/or receiving antennas can have a meander-shaped configuration. The antenna may be applied to a substrate, which particularly advantageously has high permittivity. In this way, the electrical length of the antenna increases, compared to the mathematical or geometric length thereof.

It is also advantageously possible to use ceramic antennas as transmitting and/or receiving antennas, where the wave runs over ceramics. The use of patch antennas is also possible.

This better defines the measured range of the antenna pair, making it less susceptible to disturbances from high permittivity bodies (such as arms or hands) entering the near field of the antenna from outside. Such directional characteristics can, for example, be achieved by a surface, connected to ground, behind the antenna, that is, distal with respect to the body. As an alternative or in addition, an antenna topology having non-unipolar radiation characteristics can be used. Advantageously, it is also possible to introduce a low-permittivity body behind the antennas, that is, distal with respect to the body. Another option is to arrange absorber material distally with respect to the body.

In an advantageous embodiment, the antennas can be differentially contacted. For this purpose, the antennas can, for example, be contacted with a twisted conductor pair having two currents, wherein the currents are phase-shifted by 180°.

The antennas can, in general, also be arranged at a distance from the body. In particular, dielectric or insulating material can be present between the antennas and the body. An explicit special case of this is that air is present between the antennas and the body.

In an optional embodiment, at least one of the transmitting antenna and/or receiving antenna can also be arranged in a surface, such as a support surface for the patient or a side element of a patient bed.

In an advantageous embodiment of the invention, the transmitting structure can be identical to the receiving structure. In this case, exactly one structure thus exists, for example exactly one antenna, which acts as the transmitting structure and as the receiving structure. This can take advantage of the fact that the resonant frequency of the antenna changes as a result of the change in permittivity of the body. This change can be determined as the signal. For example, a frequency that varies over time can be applied to the antenna, and it can be determined in each case at what frequency resonance exists. From the resonance, it is then possible to determine the respiratory parameter. As an alternative, it is possible to apply a frequency at which resonance exists at a distance occurring during the respiration process, so as to then determine the change in the resonance or the change in the phase over the time. The respiratory parameter can also be determined therefrom. It is in particular advantageous here to use an antenna having directional characteristics, as described above, so as to irradiate only into the body to be measured.

Advantageously, it is possible to place two insulated conductors around the thorax of the patient, for example as circumferential conductors. In the process, one conductor is guided ventrally, for example across the chest region, and one conductor is guided dorsally, for example across the back region. The thorax of the patient can thus be arranged in the manner of a dielectric between a conductor pair. Due to respiration, the permittivity of the thorax changes, and with this the propagation time of the electromagnetic waves running through the thorax, guided by the conductor. This can be detected as a change of the phase over the time. It is advantageous in the process to apply the conductor pair differentially, that is, the voltage rises at one conductor, while the voltage decreases at the second conductor to the same extent, and vice versa, as soon as polarity of the alternating field reverses. In addition, it is advantageous to shield the conductors on one side so as to reduce disturbances from the outside.

In an advantageous embodiment, it is also possible to provide information about the respiration (inspiration or expiration) via the attenuation of the wave (both transmitted (S21 parameter) and reflected (S11 parameter)). In addition to the phase information, the attenuation can be measured as an additional parameter that supports the determination of the respiratory parameters, or the attenuation can be detected independently by the device according to the invention and used for the determination of at least one respiratory parameter.

The measuring device according to the invention moreover comprises a comparison unit with which a phase of a signal supplied from the receiving structure can be compared to a phase of the AC voltage applied to the transmitting structure. The fact that the receiving structure supplies the signal can optionally be understood to mean that the receiving structure generates the signal, wherein the receiving structure, however, does not need to be an active element. It may also be understood to mean that the signal is received from the receiving structure, within the meaning that the signal is received originating from the receiving structure. The comparison unit can be electrically coupled to the receiving structure for this purpose. Advantageously, a comparison of the phases between signals received from at least two receiving structures is possible.

According to the invention, the measuring device furthermore comprises an evaluation unit with which at least one respiratory parameter can be determined from a result of the comparison of the phase of the signal received from the receiving structure to the phase of the AC voltage and/or the phase of an alternating electromagnetic field generated by the transmitting structure.

The measuring device is preferably configured to carry out a method according to the invention for measuring at least one respiratory parameter. In this method, an alternating electromagnetic field is irradiated, via at least one transmitting structure, into a body, for example of a person, an animal or a dummy, in that an AC voltage is generated by a signal generator which is applied to the at least one transmitting structure. The alternating field irradiated into the body is received on a, preferably opposite, side of the body by at least one receiving structure after having passed through the body. In the process, the opposite side can preferably by any side that is located opposite, with respect to the body's axis, the side from which the alternating electromagnetic field is irradiated. In a comparison step, a phase of a signal received from the at least one receiving structure is then compared to a phase of the AC voltage as a function of time, and at least one respiratory parameter is determined from a result of this comparison. Optionally, it is also possible to arrange the transmitting or receiving structure at the left of the thorax, and correspondingly to arrange the receiving or transmitting structure at the left of the abdomen, and correspondingly to arrange the receiving or transmitting structure at the right of the abdomen so as to have the signal pass through the lungs and the diaphragm.

The transmitting structure can advantageously be connected to the signal generator via a cable. In this case, the phase of the AC voltage is preferably measured at a resistor, via which the cable is fed. Likewise, the receiving structure can be connected to the comparison unit via a cable. It is then advantageously possible to measure the phase of the signal received from the receiving structure at a terminator, which is advantageously connected between the comparison unit and a reference potential. Advantageously, the phase of the output of the signal generator, or a reference defined therefor or a temporally constant reference, is compared to the phase of the reception-side input of the comparison unit, or a reference defined therefor or a temporally constant reference.

The fact that the phase of the signal received from the receiving structure is compared to the phase of the AC voltage or of the irradiated alternating field preferably means that a difference of these phases is found.

It is particularly preferred in the process that this phase difference between the phase of the received signal and the phase of the AC voltage or of the irradiated signal can be determined as a function of time. This phase difference is thus preferably determined for several non-coinciding points in time, and the respiratory parameter is then determined from a chronological progression of the phase difference.

Advantageously, a propagation time of the electromagnetic wave through the body can be inferred from the comparison of the phases of the AC voltage and of the received signal. Such propagation times are also preferably determined as a function of time, so that the at least one respiratory parameter can be ascertained from the chronological progression of the propagation time.

The phase or the propagation time corresponds to the instantaneous expansion of the body between the transmitting structure and the receiving structure, as well as optionally to a change in dielectric properties of the volume between the transmitting structure and receiving structure. The alternating electromagnetic field is preferably irradiated into a thorax and/or an abdomen of the body via the at least one transmitting structure. For this purpose, the at least one transmitting structure can be attached to the thorax or the abdomen. The alternating field is then preferably received by the at least one receiving structure on the opposite side of the thorax and/or of the abdomen. In the process, the transmitting structure and the receiving structure are particularly preferably attached laterally at the abdomen and/or at the thorax so that the electromagnetic wave passes laterally through the body.

In these cases, the phase or the propagation time corresponds to the instantaneous expansion of the lungs (including diaphragm, costal arch, and the like). When the expansion of the lungs changes as a result of breathing, the phase difference or the propagation time also changes. The change in the phase difference or the change in the propagation time is based, on the one hand, on the expansion of the body or of the chest during inspiration and, on the other hand, on the change in the dielectric properties of the region of the body through which the electromagnetic wave passes. On the one hand, tissue and organs in the body shift and, on the other hand, the volume of air in the lungs changes, thereby changing the electrical properties.

Advantageously, a depth of the breath of the person, the animal or the phantom can be derived, for example, via a phase deviation, that is, the difference between the minimum phase difference and the maximum phase difference in a selected time window. In this case, the at least one respiratory parameter can be a volume of a breath of air of the person, the animal or the dummy.

In an advantageous embodiment of the invention, the irradiated alternating field or the AC voltage applied to the transmitting structure can be generated with variable and/or varying frequency. The at least one respiratory parameter can then advantageously be determined at one of the multitude of frequencies at which the coupling between the irradiated alternating field and the received alternating field, or between the transmitting structure and the receiving structure, is maximal and/or at which an amplitude of the received alternating field or of the signal received from the receiving structure is maximal. It is also possible to select the frequency for the determination of the respiratory parameter at which, over the course of a breathing cycle, a maximum change results in the phase difference between the phase of the signal received from the receiving structure and the phase of the AC voltage. In this way, an optimal measuring frequency can be ascertained. The optimal measuring frequency can, in particular depending on the body size, be different for different persons, animals or phantoms.

Advantageously, it is also possible to measure simultaneously at multiple frequencies, and to compare the values of the at least one respiratory parameter, which are determined at the different frequencies, to one another or to offset these against one another, for example by means of averaging. Furthermore, it is optionally possible to transmit the frequencies in short chronological succession in a time division multiplexing process, or to transmit the frequencies simultaneously in a frequency division multiplexing process, and to use the individual measuring frequencies for conducting a plausibility check, for example regarding the assessment of the temporal gradients thereof. This plausibility check can be used, among other things, to preclude movement artefacts from the measuring series. Furthermore, it is optionally possible to use the different measuring frequencies by way of adaptive filtering techniques, correlation filters or methods of machine learning for enhancing the signal quality.

In an advantageous embodiment of the invention, the at least one transmitting structure can be connected to the signal generator via a first cable. In the process, the first cable preferably has a predefined wave impedance. The transmitting structure is then preferably fed via a first resistance, the value of which is equal to the wave impedance of the first cable. The at least one receiving structure is advantageously connected to the comparison unit via a second cable, wherein the second cable preferably has a predefined wave impedance. The receiving structure is then preferably terminated via a second resistance, the value of which is equal to the wave impedance of the second cable. For example, the first and second resistances as well as the wave impedance of the first and second cables can be 50 ohm. Optionally, the wave impedance of the transmitting structure can also be equal to the wave impedance of the cable. The same preferably applies to the wave impedance of the receiving structure.

Advantageously, the AC voltage and/or the irradiated alternating field are generated with a frequency of greater than or equal to 10 MHz, preferably greater than or equal to 30 MHz, preferably greater than or equal to 100 MHz, and/or smaller than or equal to 1000 MHz, preferably smaller than or equal to 500 MHz, and preferably smaller than or equal to 300 MHz.

In an advantageous embodiment of the invention, two, three, four or more than four transmitting structures can be provided and/or two, three, four or more than four receiving structures can be provided. In this way, more precise measurement results can be achieved. In the event that multiple transmitting structures and multiple receiving structures are used, time division multiplexing of all transmitting structures is possible to find an optimal transmitting structure-receiving structure pairing.

In the event that one transmitting structure and multiple receiving structures are provided, the results that are ascertained through the signals of the different receiving structures can be used to check the plausibility of the result. If, for example, a gradient is very high on all receivers, this may mean that the measured body is in motion. In this case, the measurement can be discarded. In the case of multiple transmitters, such plausibility checks can also take place in the time division multiplexing process. If the temporal resolution of the determination of the phase difference is selected sufficiently high, it is also possible to consider a temporal offset of the different reception signals. If the temporal relation of the individual measurement values changes drastically, this may likewise indicate a movement of the body. Furthermore, it is optionally possible to use the different reception signals by way of adaptive filtering techniques, correlation filters or methods of machine learning for enhancing the signal quality.

A combination of two transmitting structures with two receiving structures, a combination of one transmitting structure with three receiving structures, and a combination of one transmitting structure with two receiving structures have proven to be particularly advantageous. If, for example, two transmitting structures and two receiving structures are used, multiplexing between the same pairs can be advantageous. One transmitting structure and multiple receiving structures are above all advantageous for the described plausibility checks since the signals of the different receiving structures can be compared to one another.

In an advantageous embodiment of the invention, it is possible to determine, as a respiratory parameter, whether inspiration or expiration is taking place. For this purpose, the gradient of the phase difference between the phase of the received signal and the phase of the irradiated alternating field can be determined, for example as a time derivative of the phase difference. The sign of the gradient indicates whether inspiration or expiration is taking place. The determination of sequences of local minima in the phase difference and local maxima in the phase difference can also be used to determine whether inspiration or expiration is present. If, for example, a local maximum follows a local minimum, it can be concluded that inspiration is present. If a local minimum follows a local maximum, it can be concluded that expiration is present. This exemplary determination can be carried out in analogous examples analogously for other reference directions and, for example, can also be made dependent on the number of wavelengths that fit in the body. For example, a start of the inspiration or expiration can be determined by forming the derivative of the phase difference with respect to time, by averaging, correlation and the like, of the rising or falling edge.

A respiratory frequency can also be determined as the respiratory parameter. This can be determined, for example, from the time interval between two maximal phase differences or two minimal phase differences, wherein the respiratory frequency is the inverse of the time interval. Likewise, it is possible to determine the time interval between the minimal/maximal and the maximal/minimal phase difference. In the process, the time interval corresponds to the duration of the particular breathing phases. Advantageously, it is also possible to determine the respiratory frequency from the time signal by way of a Fourier transform. All that is needed here is to ascertain the rate having the greatest absolute value from the spectrum determined by way of the Fourier transform.

A tidal volume can also be determined as the respiratory parameter. The tidal volume correlates with the difference between the local minimum and the local maximum of the phase difference. In the simplest case, the correlation of the tidal volume with this difference can be assumed to be linear. For more precise determinations, the function between the tidal volume and the aforementioned difference can be analytically approximated or be experimentally measured.

In an advantageous embodiment of the invention, the method according to the invention can be used to control mechanical ventilation. Mechanical ventilation brings about a regular breathing cycle, which means that the difference between the phase of the irradiated alternating field and of the phase of the received alternating field progresses regularly. If the mechanically ventilated person carries out spontaneous breathing, this regular cycle is interrupted in a characteristic manner. The method according to the invention can then advantageously be used to send a signal to the respiratory apparatus which starts the inspiration or expiration. The respiratory apparatus can then accordingly start an advantageously supporting inspiration (supply of air) or expiration (interruption in the mechanical ventilation), so as to support the person and not providing ventilation against spontaneous breathing. The different reception signals can, for example, be considered in the computation by way of adaptive filtering techniques, correlation filters, or methods of machine learning.

The method according to the invention or the device according to the invention can also be used in a device for injecting a contrast agent (for example for CT/MRI/ultrasound) so as to optimally adapt the injection of the contrast agent to the respiration. In the process, the injection can advantageously take place when the patient is just beginning the expiration, or beginning the inspiration, or holding his or her breath.

The method according to the invention or the device according to the invention can also be used for an imaging process, such as CT, X-ray, CBCT, MRI, ultrasound, so as to record the respiratory parameters (for example inspiration, expiration, instantaneous thoracic and/or abdominal expansion) while the image is being taken. In this way, for example, a fusion of the different partial images can be optimized, in a scanning imaging process, to the effect that either only partial images having the same respiratory parameter are joined to form an overall image, or a partial image is only recorded when the respiratory parameter is suitable (for example, only at the end of the expiration or at the end of the inspiration or at a defined state), or the chronologically progression of the respiratory parameters during the recording of the partial and/or overall image is used to correct the image data or is used for a combined evaluation.

In an advantageous embodiment of the invention, the alternating field can, preferably in chronological succession, be applied using a multitude of different frequencies. It is then advantageously possible to determine the at least one respiratory parameter from a comparison of the phase of the received alternating field to the phase of the irradiated alternating field at least two of the applied frequencies. The results thus obtained can be compared to one another and/or be offset against one another so as to obtain a final value of the particular respiratory parameter.

In an advantageous embodiment of the invention, a temporal change of the difference between the phase of the received alternating field and of the phase of the irradiated alternating field can be represented as a function of the time, for example in a graphical representation. This makes it possible to observe a change of the at least one respiratory parameter over the time. In this way, it is possible, for example, to carry out long-term tests (for example to monitor a trend), and to transmit direct feedback.

The AC voltage is preferably applied to the transmitting structure in such a way that a current potentially flowing through the body is smaller than or equal to the permissible patient auxiliary current, for example smaller than or equal to 100 μA.

In an advantageous embodiment of the invention, at least one further electrode and/or at least one further measuring frequency can be used and evaluated, so as to determine at least one disturbance variable, such as an influence of a heart beat and/or a movement of the body, and remove it from the computation of the respiratory parameter. In this way, the influence of the heart beat can, for example, be extracted from the signal using a scaled Fourier linear combiner or using other adaptive filtering techniques. Advantageously, it is also possible to remove at least one disturbance variable from the computation of the at least one respiratory parameter determined in the method, for example by means of a frequency filter, an adaptive filter, a correlation filter, and/or a smoothing filter and/or a derivative of the signal. For example, a moving average or other smoothing methods, such as Savitzky-Golay, are possible here.

The measured signals can also be correlated with other measurement signals, such as values of the instantaneous respiration state (inspiration, expiration), pressure and/or flow supplied or measured by a respiratory apparatus or the periphery thereof.

The method according to the invention can be carried out as a non-diagnostic method in many advantageous embodiments. It can be used, for example, to scientifically better understand respiratory behavior. For this purpose, the respiratory behavior or the at least one respiratory parameter can be monitored by means of the method according to the invention while the test subject carries out certain tasks or is exposed to certain stresses. In an advantageous application of the method according to the invention, the body may also be the body of a phantom, such as is used, for example, for breathing exercises during first aid classes or during crash tests. Using the method according to the invention, it is possible to examine the effect of breathing activities or of forces acting from the outside on such bodies.

According to the invention, furthermore a respiratory apparatus is provided, which is configured to carry out a method as described above, and to then control the respiration based on the at least one respiratory parameter. Such a respiratory apparatus can also be used during diving, for example.

The invention will be described hereafter by way of example based on several figures. Identical reference numerals denote identical or corresponding features. The features described in the examples can also be implemented independently of the specific example and be combined between the examples.

In the drawings:

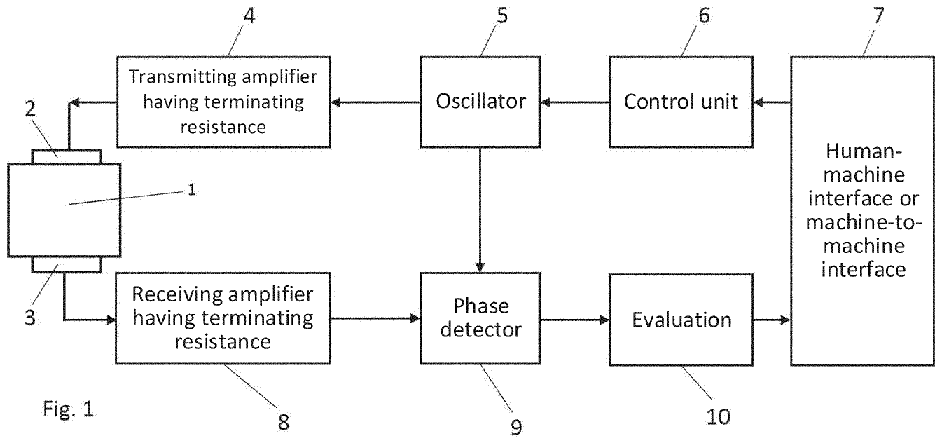

FIG. 1 shows a basic design of a measuring device according to the invention in the form of a block diagram;

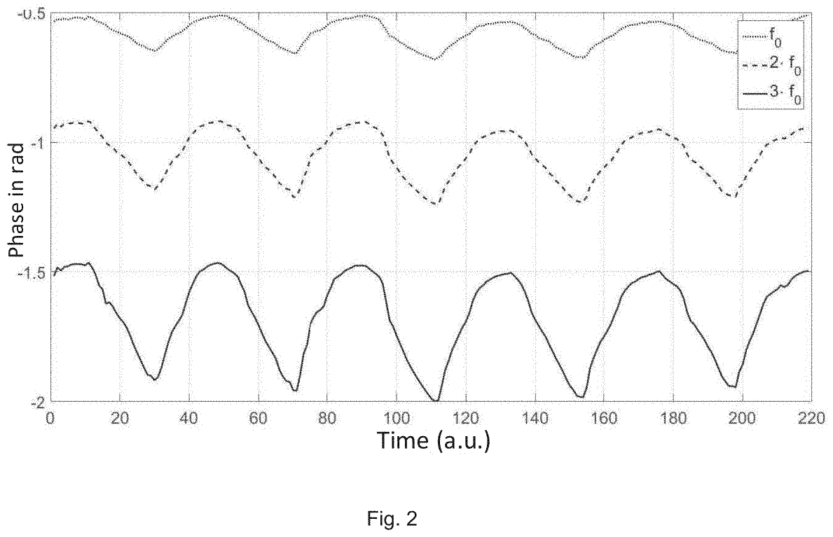

FIG. 2 shows a curve of a phase of a received signal at different measuring frequencies;

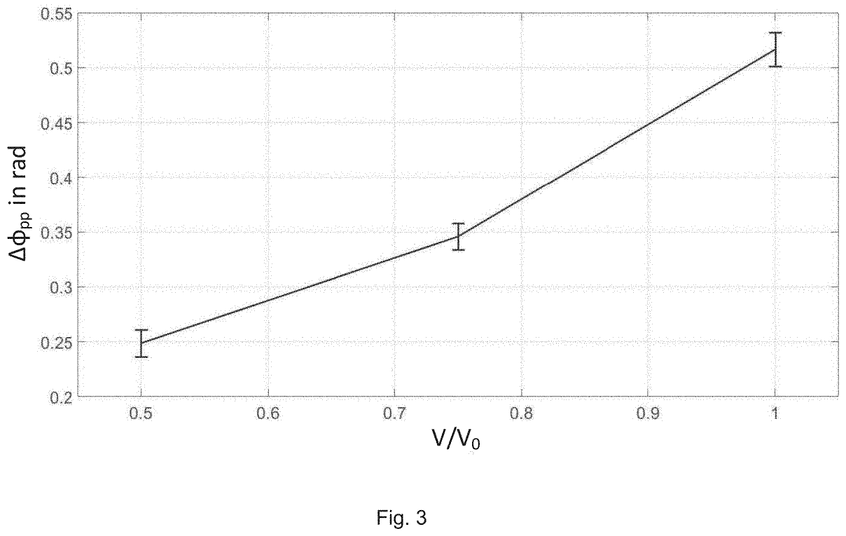

FIG. 3 shows a phase deviation for different breathing volumes;

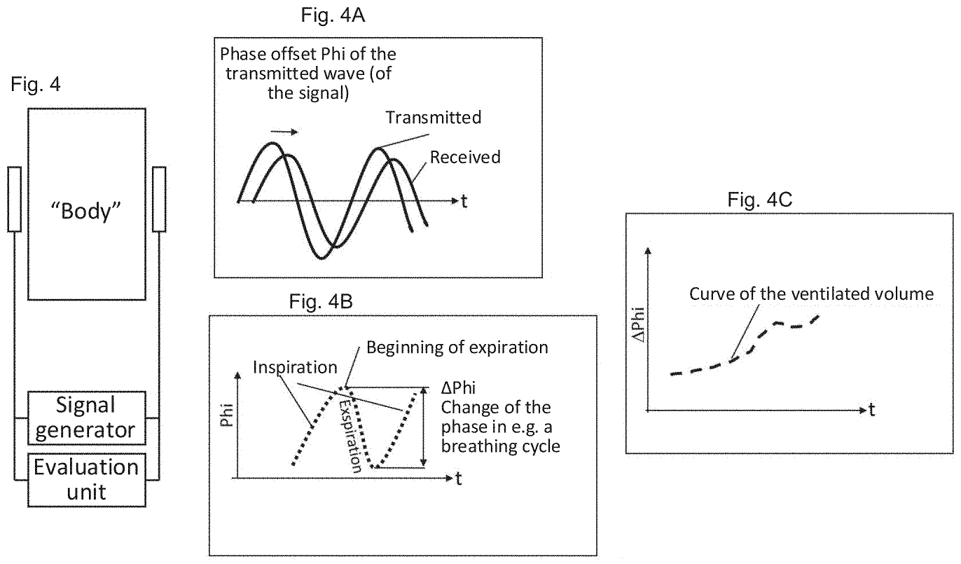

FIG. 4 shows, by way of example, the determination of a phase difference between an irradiated signal and a received signal as well as an exemplary determination of respiratory parameters;

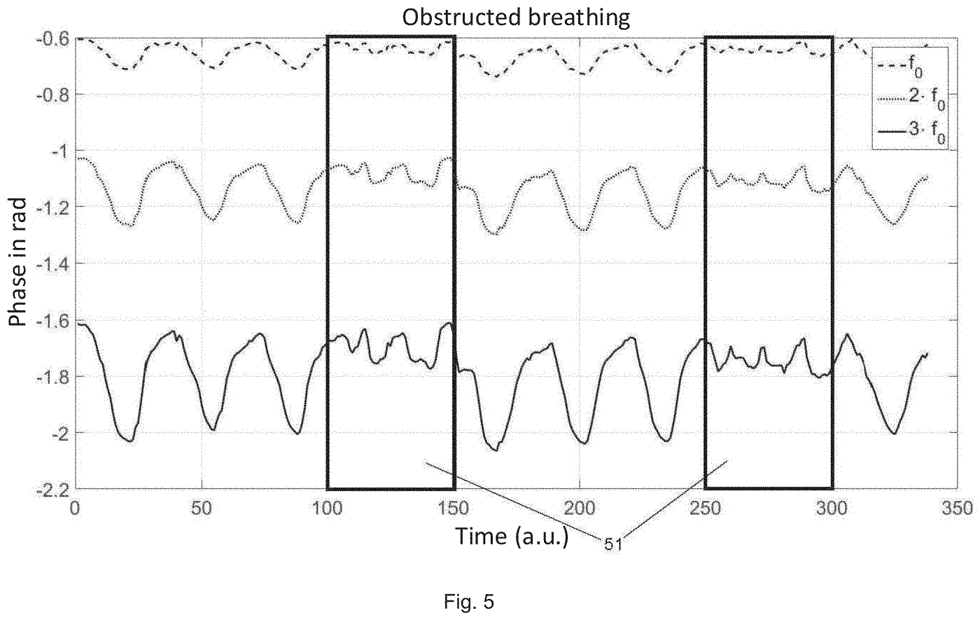

FIG. 5 shows a phase difference over the time with unobstructed and obstructed breathing;

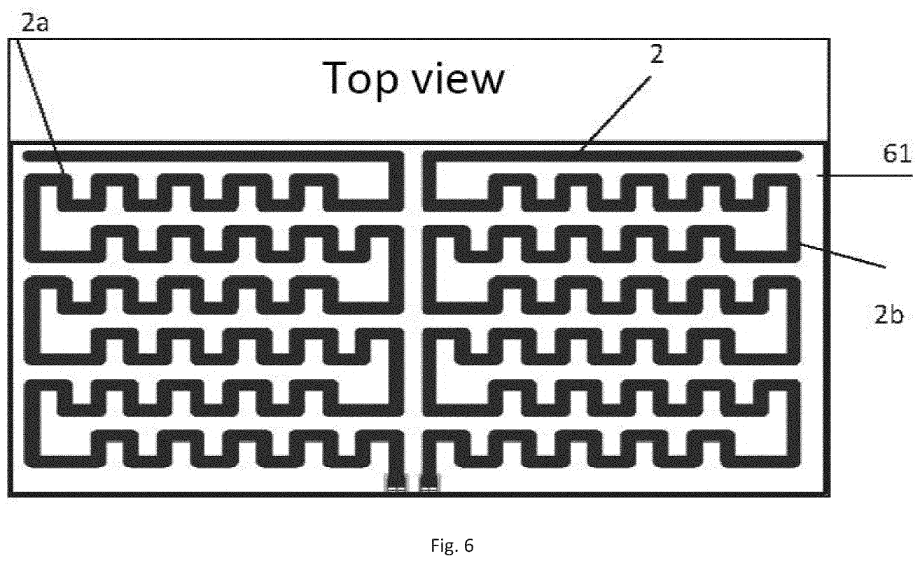

FIG. 6 shows a meander-shaped antenna structure;

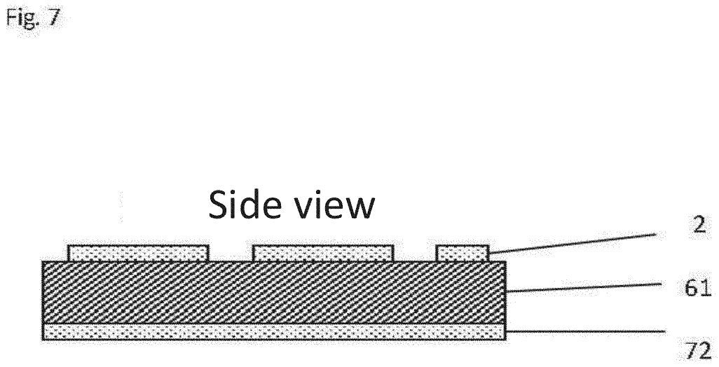

FIG. 7 shows the antenna structure shown in FIG. 6 in a side view;

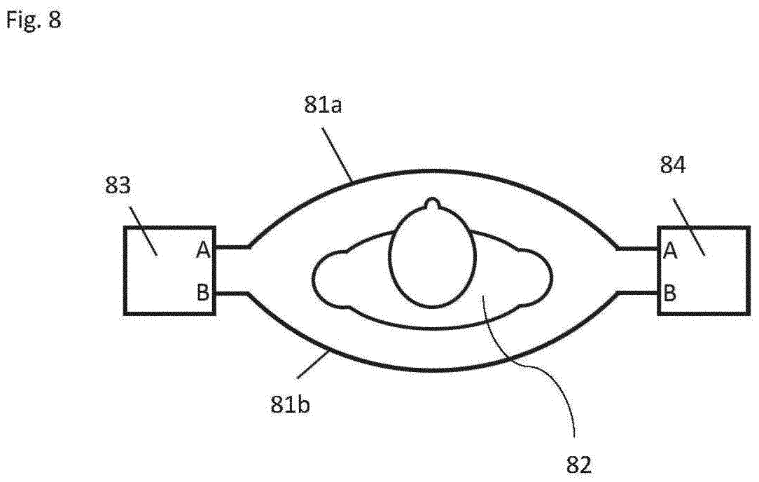

FIG. 8 shows an embodiment of the transmitting and receiving structures in the form of circumferential conductors; and

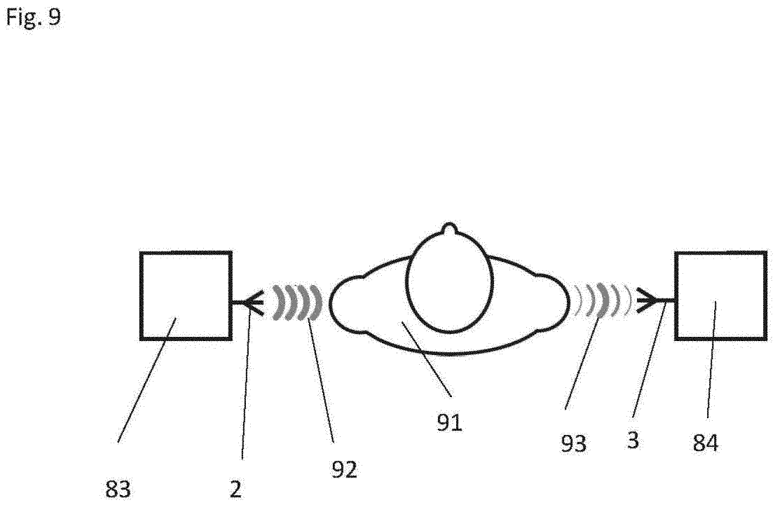

FIG. 9 shows an embodiment of the transmitting and receiving structures arranged at a distance from the patient.

FIG. 1 shows an exemplary design of a measuring device according to the invention in the form of a block diagram. A respiratory parameter is determined in the process by irradiating an alternating electromagnetic field through a body 1. The alternating electromagnetic field is irradiated into the body 1 by a transmitting structure 2, which can be an antenna or an electrode, for example, and is received by a receiving structure 3, which likewise can be an antenna or an electrode. So as to generate the signal to be irradiated, an AC voltage, which is supplied via a cable here, for example a coaxial cable, is applied to the transmitting electrode 2. Such a cable can have a defined wave impedance. The AC voltage is amplified in the process by a transmitting amplifier 4 having a terminating resistance, wherein the value of the terminating resistance is preferably equal to the wave impedance of the cable via which the transmitting electrode 2 is connected to the transmitting amplifier 4.

The AC voltage is supplied to the transmitting amplifier 4 via an oscillator 5, which generates the AC voltage with a given frequency and a certain phase. For this purpose, the oscillator 5 is controlled by a control unit 6, which can be controlled by a suitable interface, for example a human-machine interface or a machine-to-machine interface.

The alternating field irradiated into the body 1 from the transmitting electrode 2 is received by a receiving structure 3, which is connected, for example via a coaxial cable, to a receiving amplifier 8 having a terminating resistance. The absolute value of the terminating resistance is preferably equal to a wave impedance of the cable via which the receiving amplifier 8 is connected to the receiving electrode 3. The receiving amplifier 8 is connected to a phase detector 9, which is able to measure a phase of the signal received by the receiving structure 3 and amplified by the receiving amplifier 8. The phase detector 9 is furthermore connected to the oscillator 5, which generates the transmission signal. The phase detector 9 receives information about the phase of the irradiated signal from the oscillator 5. The phase detector 9 can thus carry out a comparison of the phase of the irradiated signal to the phase of the detected signal and, for example, determine a phase difference between these signals. This phase difference is particularly preferably determined as a function of time, that is, for at least two or more points in time. The phase detector can then send the, preferably time-dependent, phase difference to an evaluation unit 10, which determines the at least one respiratory parameter from the phase difference. The evaluation unit 10 can carry out suitable computing or correction steps for this purpose, such as averaging, differentiation, determination of minima and maxima, adaptive filtering, correlation filtering, frequency filtering, and the like. The evaluation unit 10 can then pass the ascertained result, that is, the respiratory parameter, on to the interface 7, where it is accessible for a person or a machine, such as a respiratory apparatus.

FIG. 2 shows the curve of the phase over time during natural breathing at three different measuring frequencies, which are represented as dotted, dashed and solid lines. The dashed line shows a measurement at double the frequency of the dotted line, and the solid line shows a measurement at triple the frequency of the dotted line. It is apparent that the measurement at triple the frequency of the dotted line shows the greatest phase deviation and is therefore suited best as the measuring frequency.

FIG. 3 shows the phase deviation for different breathing volumes. The phase deviation is plotted on the vertical axis, and the breathing volumes are plotted relative to a reference volume on the horizontal axis. It is apparent that an approximately proportional relationship exists between the breathing volume and the phase shift. The greater the breathing volume, the greater is the phase shift.

FIG. 4, by way of example, shows how a progression of the breathing volume can be determined from a sent and a received signal. The time curves of the transmitted signal (larger amplitude) and of the received signal (smaller amplitude) are plotted in partial FIG. 4A. A phase offset Phi exists between the transmitted signal and the received signal, which is the temporal difference between identical phases, such as the maximum or the minimum, of the transmitted signal and the received signal, multiplied by the angular frequency.

This phase offset Phi, also referred to as phase difference, is plotted against the time in seconds in partial FIG. 4B. A progression of the phase offset as shown by the dotted line is obtained. Shown by way of example here, the phase offset increases during inspiration. The phase offset decreases during expiration. The inverse of the distance with adjoining maximum or minima is the respiratory frequency.

FIG. 4C shows a chronological progression of the phase offset or the phase difference in relation to the time. Here, ΔPhi is plotted, which is the difference between the maximum phase offset Phi and the minimum phase offset Phi, as is plotted in FIG. 4B. Since the breathing volume increases over time, ΔPhi increases over time.

FIG. 5 shows the curves of a phase measured in the method according to the invention over time, with sections of unobstructed and obstructed breathing. An obstructed phase string is apparent in the sections denoted by reference numeral 51. The phase deviation, that is, the difference between the maximum phase difference and the minimum phase difference, is considerably smaller here during a breath than in the case of unobstructed breathing. In this way, the method according to the invention can be used to identify an obstruction. Furthermore, labored breathing during the obstruction is also apparent, which can be used, for example, for controlling mechanical ventilation.

FIG. 6 shows an example of an antenna structure 2 arranged on a substrate 61. The substrate 61 can have high permittivity here, whereby an electrical length of the antenna is increased compared to the geometric length thereof. The antenna here is designed as a dipole antenna, the mechanical width of which is shortened in relation to the electrical length thereof (that is, the length of the conductors 2a and 2b), by placing the dipole arms 2a and 2b in a meander-shaped manner.

FIG. 7 shows the embodiment of the antenna shown in FIG. 6 in the side view, viewed in the direction parallel to the surface of the substrate 61. In this example, the antenna 2 is arranged on a substrate 2 which, on the side thereof facing away from the antenna 2, is arranged on a ground plane 72. The ground plane can shield the antenna against rear-side disturbances.

FIG. 8 shows an exemplary embodiment in which two insulated conductors 81a, 81b, serving as antennas, are placed around the thorax of the patient 82. In the process, one conductor 81a is guided ventrally, for example across the chest region, and one conductor 81b is guided dorsally, for example across the back region.

A transmitting structure 83, here serving as a differential interpretation comprising a terminal A and a terminal B which can be activated in phase opposition, the receiving structure 84 (likewise differential), as well as the ventrally guided conductor 81a and the dorsally guided conductor 81b are shown here. The voltage can be applied here between the terminals A and B. If, for example, the impedance is then measured, changes in the body of the person 82 can then be inferred therefrom.

FIG. 9 shows an arrangement in which the transmitting and receiving antennas 2, 3 are arranged at a distance from the body of the person 91. An alternating field 92 is emitted here from the transmitting structure 83, and a field 93 modulated by respiration is received by the receiving structure 84. The antennas are arranged at a distance from the body of the person 91. The alternating fields 92, 93 thus pass through a region in the air.

The method according to the invention is advantageous compared to conventional methods since the measurement can take place directly, and no adjustment of the signal for a cardiac component is required. The cardiac components, however, can be taken into consideration so as to increase the signal quality. Furthermore, it is possible to minimize artefacts and a drift of the measurement signal as a result of a change in the electrical conductivity of the electrodes or the skin, since this does not involve a determination of the conductivity. Moreover, the method according to the invention is very low in movement artefacts, which would occur if amplitudes were considered alone. Compared to the bands around the chest, the wearing comfort for the user is considerably higher due to a low number of attached electrodes and/or antennas. It is also possible to use existing ECG electrodes.

The described transmission measurement of the invention allows the measured volume to be clearly defined, which is advantageous over reflecting measurements in which the volume is less clearly defined since the penetration depth is dependent on the dielectric properties, and thus on the structure of the measured tissue. The method according to the invention allows spontaneous breathing during mechanical ventilation to be identified. The measurements are intrinsically low in movement artefacts, whereby little post-processing and no additional sensor system are required, but may be used to enhance the signal quality. Compared to other measurements, the user is less restricted.

1-26. (canceled)

27. A measuring device for determining at least one respiratory parameter, comprising:
at least one transmitting structure and at least one receiving structure;
a signal generator, which is coupled to the at least one transmitting structure and with which an AC voltage can be generated that can be applied to the at least one transmitting structure;
a comparison unit, with which a phase of a signal supplied from the receiving structure can be compared to a phase of the AC voltage as a function of time; and
an evaluation unit, with which at least one respiratory parameter can be determined from a result of the comparison of the phase of the signal supplied by the receiving structure to the phase of the AC voltage.

28. The measuring device according to claim 27, wherein a phase difference between the phase of the signal supplied by the receiving structure and the phase of the AC voltage can be determined with the comparison unit as a function of time.

29. The measuring device according to claim 27, wherein the AC voltage can be generated with variable and/or varying frequency.

30. The measuring device according to claim 27,
wherein the at least one transmitting structure is connected to the signal generator via a first cable having a predefined wave impedance and is fed via a first resistance, the value of which is equal to the wave impedance of the first cable,
and wherein the receiving structure is connected to the comparison unit via a second cable having a predefined wave impedance and is terminated via a second resistance, the value of which is equal to the wave impedance of the second cable,
and optionally, the first and second resistances as well as the wave impedance of the first and second cables are 50 ohms.

31. The measuring device according to claim 27,
wherein the signal generator is configured to generate the AC voltage with a frequency of greater than or equal to 10 MHz.

32. The measuring device according to claim 27,
wherein the measuring device comprises three, four or more than four of the transmitting structures and/or three, four or more than four of the receiving structures.

33. The measuring device according to claim 27, wherein the at least one transmitting structure is an antenna or an electrode, and/or wherein the at least one receiving structure is an antenna or an electrode.

34. The measuring device according to claim 33, wherein at least one of the antennas of the receiving structure is arranged in the near field of at least one antenna of the transmitting structure.

35. The measuring device according to claim 33, wherein the at least one transmitting and/or receiving antenna has a meander-shaped structure, and optionally, the at least one transmitting and/or receiving antenna is applied to a substrate having increased permittivity.

36. A method for determining at least one respiratory parameter, wherein:
an alternating electromagnetic field is irradiated, via at least one transmitting structure, into a body by generating an AC voltage with a signal generator, which AC voltage is applied to the at least one transmitting structure;
the alternating field irradiated into the body is received by at least one receiving structure after having passed through the body;
in a comparison step, a phase of a signal supplied from the at least one receiving structure is compared to a phase of the AC voltage as a function of time; and
at least one respiratory parameter is determined from a result of the comparison step.

37. The method according to claim 36, wherein, in the comparison step, a phase difference between the phase of the signal supplied by the receiving structure and the phase of the AC voltage is determined as a function of time.

38. A method for determining at least one respiratory parameter, wherein:
an alternating electromagnetic field is irradiated, via at least one transmitting structure, into a body by generating an AC voltage with a signal generator, which AC voltage is applied to the at least one transmitting structure;
a change in a resonance of the transmitting structure during the irradiation of the alternating electromagnetic field is determined as a function of time, and a respiratory parameter is determined therefrom.

39. The method according to claim 36,
wherein the at least one transmitting structure is arranged on a thorax and/or an abdomen of the body, and
the at least one receiving structure is attached to the thorax and/or the abdomen.

40. The method according to claim 39, wherein the transmitting structure and the receiving structure are attached laterally at the abdomen and/or at the thorax.

41. The method according to claim 36,
wherein a maximum phase deviation is determined from the comparison of the phase of the signal supplied by the at least one receiving structure to the phase of the AC voltage, and a volume of a breath of air is determined as the at least one respiratory parameter from the maximum phase deviation.

42. The method according to claim 36, wherein a derivative of the difference between the phase of the signal supplied by the at least one receiving structure to the phase of the AC voltage is determined, and whether inspiration or expiration is taking place is determined as the respiratory parameter from the derivative.

43. The method according to claim 36,
wherein the AC voltage is generated, having a plurality of different frequencies, and the at least one respiratory parameter is determined from a comparison of the phase of the signal supplied by the at least one receiving structure to the phase of the AC voltage at one of the frequencies at which, among all of the plurality of frequencies, the coupling between the transmitting structure and the receiving structure is maximal, or an amplitude of the signal supplied by the receiving structure is maximal, or a maximum change in the difference between the phase of the signal supplied by the receiving structure and the phase of the AC voltage results over the course of a breathing cycle.

44. The method according to claim 36,
wherein the AC voltage is generated, utilizing a plurality of different frequencies, and the at least one respiratory parameter is determined from a comparison of the phase of the signal supplied by the receiving structure to the phase of the AC voltage at least two of the applied frequencies.

45. The method according to claim 36,
wherein a temporal beginning and/or a progression of an inspiration and/or expiration, a blockage of the respiratory tract and/or labored breathing against mechanical ventilation is determined as the at least one respiratory parameter.

46. The method according to claim 36, wherein a temporal change in a difference between the phase of the signal supplied by the receiving structure and the phase of the AC voltage is represented as a function of time.

47. The method according to claim 36, wherein at least one further electrode and/or at least one further measuring frequency is evaluated so as to remove at least one disturbance variable from the computation.

48. The method according to claim 36, wherein at least one disturbance variable is removed from the computation of a respiratory parameter determined in the method.

49. The method according to claim 36, which is a non-diagnostic method.

50. A respiratory apparatus, wherein the respiratory apparatus is configured to carry out a method according to claim 36, and to control the respiration based on the at least one respiratory parameter.

51. A device for injecting a contrast agent for imaging processes, which is configured to carry out a method according to claim 36 so as to carry out the injection of the contrast agent as a function of the respiratory parameter.

52. A device for imaging, which is configured to carry out a method according to claim 36 so as to only record images at defined respiratory parameters and/or so as to only use collected image data at defined respiratory parameters and/or so as to offset collected image data in the computation as a function of the respiratory parameters.
